(19) United States
(12) Patent Application Publication
(10) Pub. No.: US 2020/0281462 A1
(43) Pub. Date: Sep. 10, 2020

(54) OPHTHALMIC IMAGING APPARATUS, CONTROL METHOD FOR OPHTHALMIC IMAGING APPARATUS, AND COMPUTER-READABLE MEDIUM

(71) Applicant: CANON KABUSHIKI KAISHA, Tokyo (JP)

(72) Inventors: Wataru Sakagawa, Kawasaki-shi (JP); Yasuhisa Inao, Tokyo (JP)

(21) Appl. No.: 16/857,034

(22) Filed: Apr. 23, 2020

Related U.S. Application Data
(63) Continuation of application No. PCT/JP2018/039174, filed on Oct. 22, 2018.

(30) Foreign Application Priority Data
Oct. 27, 2017 (JP) 2017-208428
Aug. 6, 2018 (JP) 2018-147777

(51) Int. Cl. A61B 3/10; A61B 3/00; G06T 11/00; G06T 7/00

(52) U.S. Cl. CPC A61B 3/102; A61B 3/0025; G06T 2207/10101; G06T 7/0012; G06T 2207/30041; G06T 11/008

(57) ABSTRACT

An ophthalmic imaging apparatus includes: a detector arranged to detect, as an interference signal, interference light resulting from returning light and reference light, the returning light being light from an object to be inspected to which measurement light is radiated, the reference light corresponding to the measurement light; a converter arranged to convert the detected interference signal that is an analog signal to a digital signal; and an arithmetic processing unit configured to generate a tomographic image of the object to be inspected by using the converted interference signal. The arithmetic processing unit uses a plurality of components obtained from the converted interference signal to generate the tomographic image, the plurality of components including a component having a frequency higher than a Nyquist frequency of the converter and a component having a frequency lower than the Nyquist frequency of the converter.

### CROSS-REFERENCE TO RELATED APPLICATIONS

This application is a Continuation of International Patent Application No. PCT/JP2018/039174, filed Oct. 22, 2018, which claims the benefits of Japanese Patent Application No. 2017-208428, filed Oct. 27, 2017, and Japanese Patent Application No. 2018-147777, filed Aug. 6, 2018, all of which are hereby incorporated by reference herein in their entirety.

### BACKGROUND OF THE INVENTION

### Field of the Invention

The present invention relates to an ophthalmic imaging apparatus, a control method for an ophthalmic imaging apparatus, and a computer-readable medium.

### Description of the Related Art

Currently, as ophthalmic apparatuses for observing and/or imaging an eye, for example, an anterior-eye imaging apparatus, a fundus camera, a confocal scanning laser ophthalmoscope (SLO) apparatus, an optical coherence tomography (OCT) apparatus using multi-wavelength light-wave interference, and so on are available. Among others, an OCT apparatus can obtain a high-resolution tomographic image of an object, and therefore, is becoming an ophthalmic apparatus specifically essential to the department of ophthalmology specializing in the retina.

In an ophthalmic OCT apparatus, in a case where the fundus of an eye to be inspected is imaged in a wide range, in a case where the fundus curves to a large degree, or in a case where a region to be imaged is long in the depth direction, the difference between the optical path length of reference light and the optical path length of irradiation light may be large. The optical path length of irradiation light means the optical path length of an optical path that is a combination of the optical path of the irradiation light and the optical path of reflected light thereof. If the optical path length difference is large, folding or chipping may occur in a tomographic image in the OCT apparatus, and it might not be possible to obtain a tomographic image that correctly represents the shape of the fundus.

Accordingly, a technique for flipping a tomographic image in which folding occurs, combining the flipped image with the original tomographic image, and detecting the retina by performing an image analysis is disclosed in Japanese Patent Application Laid-Open No. 2014-176566. This technique enables an appropriate shape analysis of the fundus in a wide range in the depth direction.

### SUMMARY OF THE INVENTION

An ophthalmic imaging apparatus according to an aspect of the present invention includes a detector, a converter, and an arithmetic processing unit. The detector is arranged to detect, as an interference signal, interference light resulting from returning light and reference light, the returning light being light from an object to be inspected to which measurement light is radiated, the reference light corresponding to the measurement light. The converter is arranged to convert the detected interference signal that is an analog a to a digital signal. The arithmetic processing unit is configured to generate a tomographic image of the object to be inspected by using the converted interference signal. The arithmetic processing unit uses a plurality of components obtained from the converted interference signal to generate the tomographic image, the plurality of components including a component having a frequency higher than a Nyquist frequency of the converter and a component having a frequency lower than the Nyquist frequency of the converter.

Further features of the present invention will become apparent from the following description of exemplary embodiments with reference to the attached drawings.

### BRIEF DESCRIPTION OF THE DRAWINGS

FIG. 1 is a schematic diagram of an OCT apparatus according to an embodiment.

FIG. 2A illustrates a certain cross section of the fundus of an object to be inspected.

FIG. 2B is a diagram illustrating a relationship between the frequency of a signal and a tomographic image according to the embodiment.

FIG. 2C illustrates a tomographic image in a certain stage.

FIG. 2D is a diagram illustrating a relationship between the frequency of a signal and a tomographic image according to the embodiment.

FIG. 2E is a diagram illustrating a relationship between the frequency of a signal and a tomograph image according to the embodiment.

FIG. 3A is a diagram illustrating a method for obtaining a tomographic image that is wider in the depth direction according to the embodiment.

FIG. 3B includes diagrams illustrating a method for obtaining a tomographic image that is wider in the depth direction according to the embodiment.

FIG. 3C includes diagrams illustrating a method for obtaining a tomographic image that is wider in the depth direction according to the embodiment.

FIG. 3D is a diagram illustrating a method for obtaining a tomographic image that is wider in the depth direction according to the embodiment.

FIG. 4 is a flowchart illustrating a flow for obtaining a tomographic image that is wider in the depth direction according to the embodiment.

### DESCRIPTION OF THE EMBODIMENTS

The technique according to the related art is a technique in which a tomographic image in which folding occurs is flipped and connected to perform a shape analysis. Therefore, this technique does not remove the folding from the tomographic image. That is, it is not possible to obtain a tomographic image that is suitable to observation of the detailed structure of a region in which folding occurs.

Accordingly, an embodiment of the present invention has been made to obtain a tomographic image that is wider in the depth direction and in which folding is reduced.

For example, an ophthalmic imaging apparatus according to an aspect of the present embodiment uses a plurality of components (for example, information including the intensity and phase) obtained from an interference signal converted by a converter, the plurality of components including a component having a frequency higher than the Nyquist frequency of the converter and a component having a frequency lower than the Nyquist frequency of the converter, to generate a tomographic image of an object to be inspected. Accordingly, a tomographic image that is wider in the depth direction and in which folding is reduced can be obtained. The present embodiment will be described below.

### OCT Apparatus

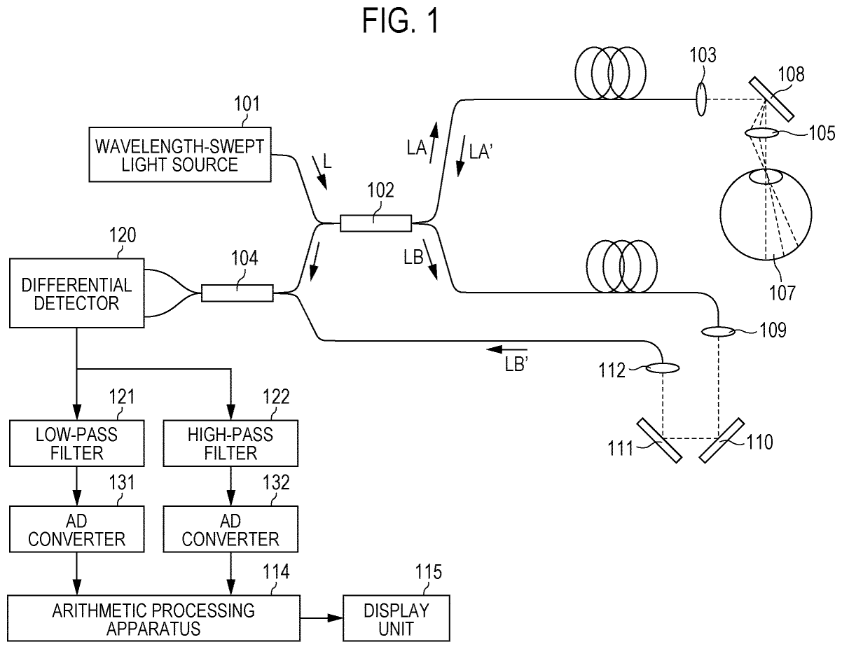

FIG. 1 is a diagram illustrating a configuration of an OCT apparatus (an example of the ophthalmic imaging apparatus) according to the present embodiment. A wavelength-swept light source 101 is a light source that emits light having a wavelength that changes from a shorter wavelength to a longer wavelength or from a longer wavelength to a shorter wavelength by time. Light L emitted from the wavelength-swept light source 101 is branched into irradiation light LA (measurement light) and reference light LB at a branching fiber coupler 102, which is an example of a separator. The irradiation light LA is collimated at a collimator lens 103, is reflected at a scanning mirror 108, passes through a condensing lens 105, and is radiated to an object to be inspected 107. Light LA′ (returning light) reflected at the object to be inspected 107 after irradiation passes through the coupler 102 and enters a coupler 104. The reference light LB enters the coupler 104 as light LB′ via a collimator lens 109, mirrors 110 and 111, and a condensing lens 112. The light LA′ and the light LB′ are combined and separated at the coupler 104 and enter a differential detector 120, which is an example of a detector, as interference light.

The differential detector 120 converts the intensity of the interference light to an analog signal. The analog signal output from the differential detector 120 is separated. The analog signal that passes through a low-pass filter 121 includes only low-frequency components, and is converted to a digital signal by an analog-digital (AD) converter 131, which is an example of a converter. The analog signal that passes through a high-pass filter 122 includes only high-frequency components, and is converted to a digital signal by an AD converter 132, which is an example of another converter. The band of the low-pass filter 121 and that of the high-pass filter 122 can be electrically adjusted. The low-pass filter 121 and the high-pass filter 122 correspond to an example of a filter unit that attenuates a detected interference signal in accordance with predetermined frequency characteristics. These digital signals are processed by an arithmetic processing apparatus 114, which is an example of an arithmetic processing unit, by using a method described below. The arithmetic processing apparatus 114 performs a process including a Fourier transform on the digital signals to obtain information about the object to be inspected 107. A display unit 115 displays a tomographic image of the object to be inspected obtained by the arithmetic processing apparatus 114. The arithmetic processing apparatus may be connected to the ophthalmic imaging apparatus so as to enable communication. The arithmetic processing apparatus may be built into the ophthalmic imaging apparatus.

The above-described process is a process for obtaining a tomographic image at a certain point of the object to be inspected 107. Acquisition of information about a cross section of the object to be inspected 107 in the depth direction is called an A-scan. A scan for obtaining information about a cross section of the object to be inspected in a direction orthogonal to the A-scan, that is, for obtaining two-dimensional information, is called a B-scan. In the present embodiment, the B-scan is performed by the scanning mirror 108.

### Acquisition of Tomographic Image

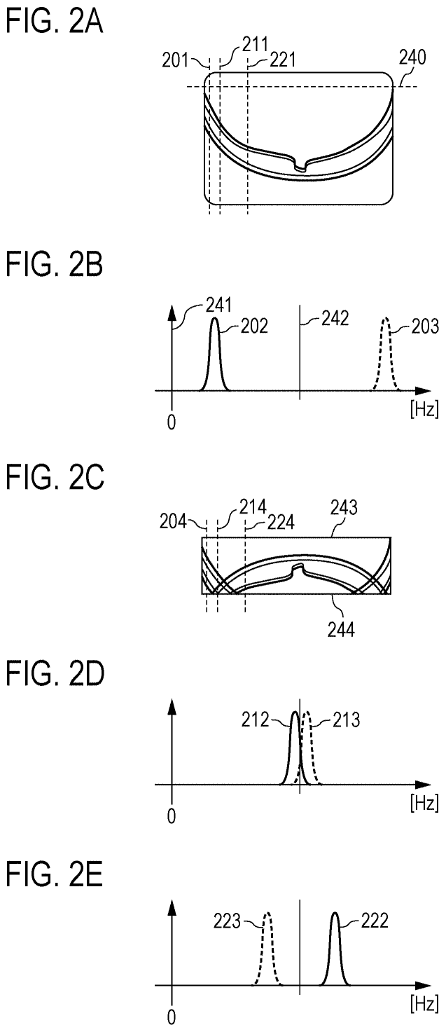

Acquisition of a tomographic image is described with reference to FIGS. 2A to 2E. Here, a case where the band of the low-pass filter 121 illustrated in FIG. 1 is adjusted so as to be sufficiently wide is described. In this case, all analog signals output from the differential detector 120 pass through the low-pass filter 121, and therefore, the low-pass filter 121 may be disregarded.

FIG. 2A illustrates a certain cross section of the fundus of the object to be inspected 107. The positions of the mirrors 110 and 111 are adjusted by using a method described below so that, in a case where irradiation light is reflected at a depth position 240, the optical path length of the irradiation light and the optical path length of the reference light match each other.

FIG. 2B indicates a signal intensity for each frequency component obtained by the arithmetic processing apparatus 114 performing a Fourier transform on a signal obtained by the A-scan at a position 201 in FIG. 2A. The horizontal axis represents the frequency, and the vertical axis represents the logarithm of the signal intensity. A DC component 241 represents a frequency 0. When the optical path length of the irradiation light and the optical path length of the reference light completely match each other, the irradiation light and the reference light intensify each other across all swept wavelengths, and a DC component signal is obtained. In a case where the optical path length of the irradiation light and the optical path length of the reference light are different from each other, interference occurs in accordance with the difference, and the frequency of the signal changes.

At the position 201, the object to be inspected 107 is located at a depth position deeper than the depth position 240, and therefore, the optical path lengths differ, and a real image 202 is obtained. A Nyquist frequency 242 is a frequency half the sampling frequency of the AD converter 131 and is the maximum frequency of a signal that can be obtained (sampled) by the AD converter 131. When analog-digital conversion is performed by the AD converter 131, folding (aliasing) occurs around an axis which is the Nyquist frequency. A mirror image 203 is an image representing a signal having a frequency in which the signal is not distinguishable from a signal of the real image 202 when sampling is performed by the AD converter 131.

FIG. 2C illustrates a tomographic image in a certain stage generated by the arithmetic processing apparatus 114. An upper end 243 of the image represents the DC component 241, and a lower end 244 of the image represents the Nyquist frequency 242. A straight line 204 visually represents the signal illustrated in FIG. 2B. The real image 202 is entirely included in a range from the DC component to the Nyquist frequency, and therefore, the tomographic image is included between the upper end and the lower end of the image on the straight line 204.

FIG. 2D indicates a signal at a position 211 in a manner similar to FIG. 2B. At the position 211, the object to be inspected 107 is located at a depth position deeper than that at the position 201, and therefore, a real image 212 has a frequency higher than that of the real image 202. The real image 212 partially goes over the Nyquist frequency and partially overlaps with a folded mirror image 213. Accordingly, on a straight line 214 that visually represents the signal illustrated in FIG. 2D, the original tomographic image and the folded tomographic image partially overlap.

FIG. 2E indicates a signal at a position 221 in a manner similar to FIG. 2B. At the position 221, the object to be inspected 107 is located at a depth position still deeper than that at the position 211, and therefore, a real image 222 has a still higher frequency and goes over the Nyquist frequency. Accordingly, on a straight line 224 that visually represents the signal illustrated in FIG. 2E, only a mirror image 223, that is, only the folding portion, is visually represented. Sampling of a signal having a frequency that exceeds the Nyquist frequency as described above is generally called undersampling.

As can be seen from the above, in a case where the sampling frequency of the AD converter 131 is not sufficient for the range of the object to be inspected 107 in the depth direction, folding occurs in the tomographic image, and it is not possible to obtain a tomographic image that correctly represents the structure of the object to be inspected 107.

The position adjustment of the mirrors 110 and 111 described above is made as follows. A user adjusts the positions of the minors 110 and 111, which correspond to an example of an optical path length changing unit, by using an input unit not illustrated while watching the image illustrated in FIG. 2C or a similar image displayed on the display unit 115. When the positions of the mirrors 110 and 111 are appropriately adjusted, a tomographic image in which folding occurs only at the lower end of the image as in FIG. 2B can be obtained. The positions of the mirrors 110 and 111 can be adjusted by, for example, moving the mirrors 110 and 111 on a common stage along the optical axis. Accordingly, the optical path length of the reference light can be changed. The optical path length changing unit may change the optical path length of the measurement light instead of the optical path length of the reference light or may change both the optical path length of the reference light and the optical path length of the measurement light. That is, the optical path length changing unit may be any unit as long as the unit can change the difference between the optical path length of the reference light and the optical path length of the measurement light.

In a case where the positions of the mirrors 110 and 111 are not appropriate, folding may occur at the upper end of the image. This is a case where the depth position 240 and the object to be inspected 107 overlap. In this case, frequency components of the signal are distributed across both sides of the DC component 241, and the negative range of the frequency is folded in the positive range of the frequency as a mirror image, which results in the occurrence of folding in the tomographic image. In an OCT apparatus according to the related art, the expression “folding in a tomographic image” sometimes refers to the above-described folding. On the other hand, the present invention assumes that the above-described folding at the upper end 243 of the image is avoided by adjusting the positions of the mirrors 110 and 111. As a result, folding due to the Nyquist frequency occurs at the lower end 244 of the image. A description is given below of a process that is additionally performed by the arithmetic processing apparatus 114 for suppressing the folding due to the Nyquist frequency and visually representing the structure of the object to be inspected 107 in a wide range in the depth direction.

For simplified description, the description given below assumes that no phase shift occurs in the filters. Further, the description given below assumes that signals obtained by the A-scan of the fundus are in phase across all frequencies. When such a signal is subjected to a Fourier transform, a frequency distribution formed of only real numbers is obtained. Therefore, only real numbers are used in the following description.

### Reduction of Folding

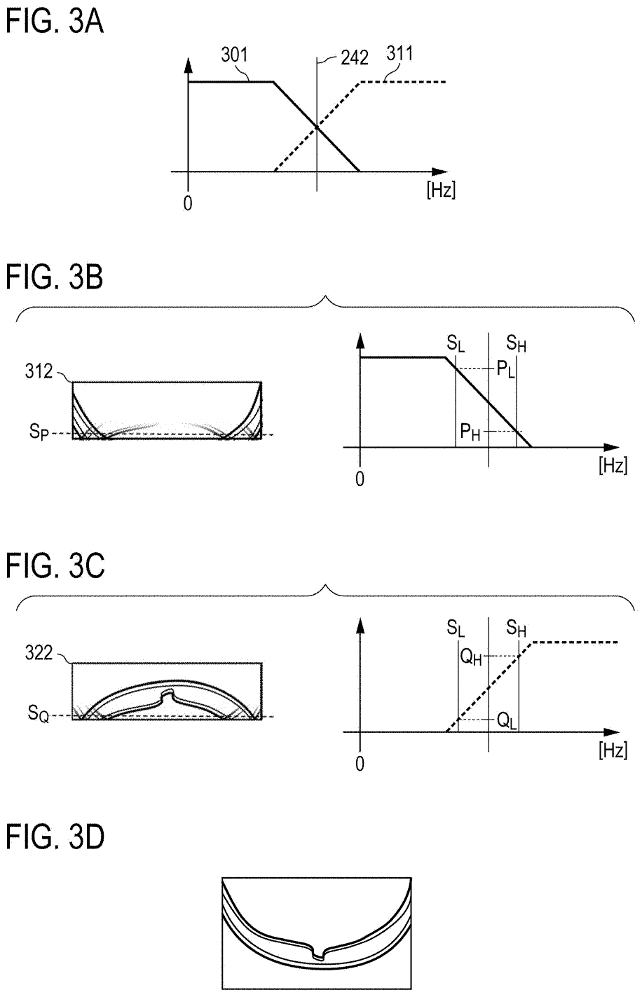

FIGS. 3A to 3D are diagrams illustrating the additional process for visually representing the structure of the object to be inspected 107 in a wide range in the depth direction. FIG. 3A is a diagram illustrating the frequency characteristics of the filters. Characteristics 301 are the frequency characteristics of the low-pass filter 121, and characteristics 311 are the frequency characteristics of the high-pass filter 122. The horizontal axis represents the frequency, and the vertical axis represents the logarithm of the filter transmittance. The both filters are adjusted so that the cutoff frequencies thereof are close to the Nyquist frequency 242. As information about the frequency characteristics of the filters, for example, a graph as illustrated in FIG. 3A or a table representing the graph can be stored in advance in a storage unit.

FIG. 3B illustrates the frequency characteristics of the low-pass filter 121 and a tomographic image 312 obtained from a digital signal generated by the AD converter 131. The tomographic image 312 is an example of a first partial tomographic image generated by using components having frequencies lower than the Nyquist frequency of the converter. A signal S_{L} and a signal S_{H} are signals output from the differential detector 120 at the respective frequencies thereof. The frequency of the signal S_{L} and the frequency of the signal S_{H} are located symmetrically about the Nyquist frequency 242. The signal S_{L} and the signal S_{H} are attenuated by the low-pass filter 121. Transmittances P_{L} and P_{H} are values indicating the transmittances of the low-pass filter 121 at the frequency of the signal S_{L} and the frequency of the signal S_{H} respectively. These values are determined when the apparatus is adjusted. The signal S_{L} and the signal S_{H} are attenuated by the low-pass filter 121, and a signal P_{L}S_{L} and a signal P_{H}S_{H} respectively resulting from the signal S_{L} and the signal S_{H} are input to the AD converter 131. When these signals are subjected to analog-digital conversion by the AD converter 131, the signals overlap due to folding and are added up, resulting in a single signal S_{P}. The arithmetic processing apparatus 114 stores the signal S_{P} obtained as a result of a Fourier transform and generates the tomographic image 312 having a luminance that is the intensity of the signal S_{P}. According to the above description, the signal S_{P} matches the result obtained by calculating the following equation.

S_{P}=P_{L}S_{L}+P_{H}S_{H}

FIG. 3C illustrates the frequency characteristics of the high-pass filter 122 and a tomographic image 322 obtained from a digital signal generated by the AD converter 132 in a manner similar to FIG. 3B. The tomographic image 322 is an example of a second partial tomographic image generated by using components having frequencies higher than the Nyquist frequency of the converter. As in FIG. 3B, a signal S_{Q} matches the result obtained by calculating the following equation. Transmittances Q_{L} and Q_{H} are values indicating the transmittances of the high-pass filter 122 at the frequency of the signal S_{L} and the frequency of the signal S_{H} respectively.

S_{Q}=Q_{L}S_{L}+Q_{H}S_{H}

When these equations are solved as simultaneous equations, the signal S_{L} and the signal S_{H} are calculated as follows.

$${S}_{L} = \frac{{Q}_{H} {S}_{P} - {P}_{H} {S}_{Q}}{{P}_{L} {Q}_{H} - {P}_{H} {Q}_{L}}$$

$${S}_{H} = \frac{{Q}_{L} {S}_{P} - {P}_{L} {S}_{Q}}{{P}_{H} {Q}_{L} - {P}_{L} {Q}_{H}}$$

Accordingly, the arithmetic processing apparatus 114 can obtain the signal S_{L} and the signal S_{H} by using the signals used to generate the tomographic image 312 and the tomographic image 322 and the transmittances of the filters. That is, the signal S_{L} having a frequency lower than the Nyquist frequency and the signal S_{H} having a frequency higher than the Nyquist frequency that overlap due to folding can be separated with the above-described method. The above-described calculation is performed for all frequencies other than the Nyquist frequency 242 to thereby obtain signals for the respective frequencies. At the Nyquist frequency 242, P_{L} and P_{H} match, and therefore, it is not possible to perform the above-described calculation. However, a signal obtained as a result of analog-digital conversion is used as is to thereby obtain the signal S_{L} (which is a signal the same as the signal S_{H} in this case).

FIG. 3D illustrates a tomographic image obtained by visually representing the intensities of the signals thus obtained to visually represent the object to be inspected 107 in a deep range. It can be seen that, unlike FIG. 2C, FIG. 3B, and FIG. 3C, a tomographic image in a wide range in the depth direction is obtained.

The description given above assumes that no phase shift occurs in the filters and assumes that signals obtained by the A-scan of the fundus are in phase across all frequencies. However, in actuality, a phase shift occurs in the frequency filters. Further, in actuality, signals obtained by the A-scan of the fundus include signals having various phases, Calculation that takes into consideration these situations can be performed. Specifically, the signals S_{L} and S_{H}, the transmittances P_{L}, P_{H}, Q_{L}, and Q_{H}, and the luminances S_{P} and S_{Q} mentioned in the above description, which are all real numbers, need to be replaced with complex numbers. The transmittances P_{L}, P_{H}, Q_{L}, and Q_{H} can be obtained as complex numbers from the gain characteristics and phase characteristics of the filters. The signals S_{P} and S_{Q} can be obtained by performing a Fourier transform on signals obtained by the A-scan of the fundus. The signals S_{L} and S_{H} are obtained from the above-described calculation as complex numbers, and the intensities thereof need to be used as the luminances of the image. Note that a component described in the present embodiment is information that includes the phase as well as the intensity and is information that includes a complex number as well as a real number.

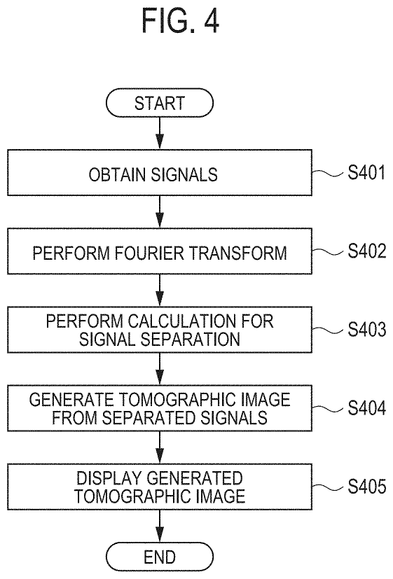

FIG. 4 is a flowchart illustrating a flow up to generation and display of a tomographic image based on the above description. In step S401, the arithmetic processing apparatus 114 obtains interference signals generated by the AD converter 131 and the AD converter 132. In step S402, the arithmetic processing apparatus 114 performs a Fourier transform on the interference signals to obtain the signals S_{P} and S_{Q}. In step S403, the arithmetic processing apparatus 114 performs calculation for separating the signals on the basis of the Nyquist frequency by using the above-described method to obtain the signals S_{L} and S_{H}. In step S404, the arithmetic processing apparatus 114 obtains the intensities of the signals S_{L} and S_{H} and generates the tomographic image illustrated in FIG. 3D. In step S405, the display unit 115 displays the tomographic image.

With the present embodiment, imaging in a short time is enabled for the following reason. It is not necessary to drive the mirrors 110 and 111 to change the optical path length of the reference light in order to obtain a plurality of images having different folding intensities, and only a single scan needs to be performed by the mirror 108 to obtain the tomographic image illustrated in FIG. 3D.

The values of the transmittances of the low-pass filter 121 and the high-pass filter 122 may be updated after an adjustment of the apparatus by taking into consideration, for example, environmental dependence. For example, the transmittances may be calculated from signals obtained from irradiation light reflected at a mirror (not illustrated) in the apparatus before and during an inspection. Alternatively, the values of the transmittances may be estimated on the basis of the luminance distribution of the obtained tomographic image illustrated in FIG. 3D, and the tomographic image illustrated in FIG. 3D may be regenerated.

Equations for calculating the signal S_{L} and the signal S_{H} may be equations other than the above-described equations. For example, in a case where the filters are close to ideal filters and the cutoff frequencies are close to the Nyquist frequency, substantially no folding portions are present in the tomographic image 312, and substantially only folding portions are present in the tomographic image 322. When the tomographic image 322 is vertically flipped and is simply connected with the tomographic image 312, a tomographic image close to a desired tomographic image can be obtained. This is equivalent to calculation in a case where P_{H} and Q_{L} are set to 0 and P_{L} and Q_{H}are set to 1 in the above-described equations.

The filters to be used need not be a combination of a low-pass filter and a high-pass filter. For example, a combination of only low-pass filters having different cutoff frequencies may be used. Even when only low-pass filters are used, if the values of the above-described P_{L} and Q_{L} are different or the values of the P_{H} and Q_{H} are different, similar calculation can be performed. Alternatively, taking into consideration frequencies higher than twice the Nyquist frequency, bandpass filters may be used.

Three or more filters may be used. Three or more filters having different frequency bands may be combined and similar calculation may be performed to obtain a tomographic image in a still deeper range. Further, signal intensities obtained by using a plurality of filters having the same bands may be averaged to increase the accuracy of luminance calculation.

The number of filters may be one. A signal that passes through the filter and a signal that does not pass through the filter may be used to perform similar calculation. In this case, the signal that does not pass through the filter is not attenuated substantially, and therefore, the values of P_{L} and P_{H} can be set to around 1. If the single filter is close to an ideal low-pass filter, Q_{L} can be set to 1, and Q_{H} can be set to 0. In this case, the above equation for S_{H} corresponds to a simple subtraction of the signal that passes through the filter from the signal that does not pass through the filter. That is, with such a configuration, a tomographic image in a wide range in the depth direction can be obtained with a signal subtraction process.

Alternatively, one filter may be used and the band may be switched by time. When a plurality of tomographic images are successively obtained while the band is switched, a tomographic image in a deeper range can be generated from the plurality of tomographic images. In this case, in order to reduce the effect of motion of an eye to be inspected, a plurality of tomographic images are obtained in a short time. For this, the band of the filter is configured to be electrically changed as in the present embodiment. As the configuration for switching the band of the filter, for example, a configuration formed of a characteristic changing unit (for example, a variable resistor) that is provided in the filter and changes the frequency characteristics of the filter and a controller that controls the characteristic changing unit may be employed.

In the present embodiment, the values of the transmittances of the filters are determined by taking into consideration attenuation of signals caused by a factor other than the filters. That is, attenuation caused by a signal intensity decrease due to defocus and attenuation caused by the characteristics of filters in the AD converters are also regarded as attenuation caused by the low-pass filter 121 and the high-pass filter 122, and calculation is performed. However, these may be handled as separate parameters.

The sampling frequency of the AD converter 131 and that of the AD converter 132 may be different from each other. The arithmetic processing apparatus 114 may be configured to perform switching as to whether the above-described process is to be performed.

The final tomographic image (FIG. 3D) may be directly generated from the obtained signals by using the above-described equations without generating the tomographic image 312 and the tomographic image 322. In this case, it is not required that the tomographic images 312 and 322 are generated and therefore higher-speed processing can be performed.

The present embodiment assumes a case where a wavelength-sweep by the wavelength-swept light source 101 is stably performed at a constant rate for the wave number; however, a light source in which the constant rate is not maintained may be used. In this case, a configuration may be employed in which a k-clock for sampling at equal wave-number intervals in the light source or in the apparatus is generated and input to the AD converters. A generator of the k-clock is an example of a clock generator that generates a clock for the converters to sample analog signals. The generator of the k-clock may be configured as an interferometer in which an optical path through which part of light from the wavelength-swept light source 101 passes is branched into a first optical path and a second optical path having an optical path length difference relative to the first optical path. Even in a case where the rate at which a wavelength-sweep by the wavelength-swept light source 101 is performed is not constant, components having frequencies higher than the Nyquist frequency of the converters are regarded as components that oscillate in a time shorter than the time in which sampling of the interference signals (analog signals) by the converters is performed twice. Even in the case where the rate at which a wavelength-sweep by the wavelength-swept light source 101 is performed is not constant, components having frequencies lower than the Nyquist frequency of the converters are regarded as components that oscillate in a time longer than the time in which sampling of the interference signals (analog signals) by the converters is performed twice.

The present embodiment is applied to a swept-source (SS)-OCT; however, the present embodiment may be applied to the other OCTs. For example, in a spectral-domain (SD)-OCT, an optical low-pass filter may be disposed forward of a line sensor to fill a role equivalent to that of the low-pass filter 121 according to the present embodiment. A decrease in resolution based on the pixel size of the line sensor or the design of a diffraction grating, lens, etc. may be regarded as an effect equivalent to that of the low-pass filter, and the present invention may be applied.

As described above, according to the present embodiment, a signal having a frequency higher than the Nyquist frequency and a signal having a frequency lower than the Nyquist frequency can be separately calculated. Accordingly, a range of frequencies higher than the Nyquist frequency, that is, a deeper range of the fundus, can be visually represented, and a tomographic image that is wide in the depth direction can be obtained. Further, signals can be obtained in a time shorter than the time taken by the method for changing the optical path length, and an effect of small fixation movement of an eye to be inspected and an effect of fatigue can be suppressed.

### Other Embodiments

Embodiment(s) of the present invention can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD™), a flash memory device, a memory card, and the like.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

What is claimed is:

1. An ophthalmic imaging apparatus comprising:
a detector arranged to detect, as an interference signal, interference light resulting from returning light and reference light, the returning light being light from an object to be inspected to which measurement light is irradiated, the reference light corresponding to the measurement light;
a converter arranged to convert the detected interference signal that is an analog signal to a digital signal; and
an arithmetic processing unit configured to generate a tomographic image of the object to be inspected by using a plurality of components relating to a plurality of signals obtained through the conversion, wherein
the arithmetic processing unit uses the plurality of components including a component having a frequency higher than a Nyquist frequency of the converter and a component having a frequency lower than the Nyquist frequency of the converter to generate the tomographic image.

2. The ophthalmic imaging apparatus according to claim 1, wherein
the arithmetic processing unit generates a first partial tomographic image by using the component having the frequency lower than the Nyquist frequency, generates a second partial tomographic image by using the component having the frequency higher than the Nyquist frequency, and combines the first partial tomographic image and the second partial tomographic image to generate the tomographic image.

3. The ophthalmic imaging apparatus according to claim 1, further comprising
a filter unit arranged to attenuate the detected interference signal in accordance with a predetermined frequency characteristic, wherein
the converter converts the attenuated interference signal.

4. An ophthalmic imaging apparatus comprising:
a detector arranged to detect, as an interference signal, interference light resulting from returning light and reference light, the returning light being light from an object to be inspected to which measurement light is radiated, the reference light corresponding to the measurement light;
a filter unit arranged to attenuate the detected interference signal in accordance with a predetermined frequency characteristic;
a converter arranged to convert the attenuated interference signal that is an analog signal to a digital signal; and
an arithmetic processing unit configured to generate a tomographic image of the object to be inspected by using a plurality of components relating to a plurality of signals obtained through the conversion, wherein the arithmetic processing unit obtains any of the plurality of components by subtraction of the plurality of signals.

5. The ophthalmic imaging apparatus according to claim 3, further comprising
another converter arranged to convert the detected interference signal, the other converter being different from the converter, wherein
the arithmetic processing unit uses the plurality of components obtained from the attenuated interference signal and from the interference signal converted by the other converter to generate the tomographic image.

6. The ophthalmic imaging apparatus according to claim 5, herein
the arithmetic processing unit obtains any of the plurality of components by subtraction of the attenuated interference signal and the interference signal converted by the other converter.

7. The ophthalmic imaging apparatus according to claim 3, further comprising:
a characteristic changing unit provided in the filter unit and arranged to change a frequency characteristic of the filter unit; and
a controller configured to control the characteristic changing unit, wherein
the arithmetic processing unit uses the plurality of components obtained from the interference signal converted before the frequency characteristic is changed and the interference signal converted after the frequency characteristic is changed to generate the tomographic image.

8. The ophthalmic imaging apparatus according to claim 3, wherein
the arithmetic processing unit obtains the plurality of components by using information about the frequency characteristic of the filter unit and the converted interference signal, and uses the obtained plurality of components to generate the tomographic image.

9. The ophthalmic imaging apparatus according to Claire 1, further comprising
an optical path length changing unit arranged to change at least one of an optical path length of the reference light and an optical path length of the measurement light, wherein
the arithmetic processing unit obtains the plurality of components in which folding components are separated from each other by using the converted interference signal obtained without changing the optical path length by the optical path length changing unit, and uses the obtained plurality of components to generate the tomographic image.

10. An ophthalmic imaging apparatus comprising:
a detector arranged to detect, as an interference signal, interference light resulting from returning light and reference light, the returning light being light from an object to be inspected to which measurement light is radiated, the reference light corresponding to the measurement light;
a converter arranged to convert the detected interference signal that is an analog signal to a digital signal;
an arithmetic processing unit configured to generate a tomographic image of the object to be inspected by using a plurality of components relating to a plurality of signals obtained through the conversion; and
an optical path length changing unit arranged to change at least one of an optical path length of the reference light and an optical path length of the measurement light, wherein
the arithmetic processing unit uses the plurality of components relating to the plurality of signals obtained through the conversion of a plurality of interference signals that are obtained without changing the optical path length by the optical path length changing unit and of which folding components differ to generate the tomographic image.

11. The ophthalmic imaging apparatus according to claim 1, further comprising
a wavelength-swept light source;
a separator arranged to separate light from the wavelength-swept light source into the measurement light and the reference light; and
a clock generator arranged as an interferometer in which an optical path through which part of the light from the wavelength-swept light source passes is branched into a first optical path and a second optical path having an optical path length difference relative to the first optical path to generate a clock for sampling by the converter.

12. A control method for an ophthalmic imaging apparatus including a detector and a converter, the detector arranged to detect, as an interference signal, interference light resulting from returning light and reference light, the returning light being light from an object to be inspected to which measurement light is radiated, the reference light corresponding to the measurement light, the converter arranged to convert the detected interference signal that is an analog signal to a digital signal, the control method comprising
using a plurality of components relating to a plurality of signals obtained through the conversion to generate a tomographic image of the object to be inspected, the plurality of components including a component having a frequency higher than a Nyquist frequency of the converter and a component having a frequency lower than the Nyquist frequency of the converter.

13. A control method for an ophthalmic imaging apparatus including a detector, a filter unit, and a converter, the detector arranged to detect, as an interference signal, interference light resulting from returning light and reference light, the returning light being light from an object to be inspected to which measurement light is radiated, the reference light corresponding to the measurement light, the filter unit arranged to attenuate the detected interference signal in accordance with a predetermined frequency characteristic, the converter arranged to convert the attenuated interference signal that is an analog signal to a digital signal, the control method comprising:
obtaining any of a plurality of components relating to a plurality of signals obtained through the conversion, by subtraction of the plurality of signals; and
using the plurality of components to generate a tomographic image of the object to be inspected.

14. A control method for an ophthalmic imaging apparatus including a detector, a converter, and an optical path length changing unit, the detector arranged to detect, as an interference signal, interference light resulting from returning light and reference light, the returning light being light from an object to be inspected to which measurement light is radiated, the reference light corresponding to the measurement light, the converter arranged to convert the detected interference signal that is an analog signal to a digital signal, the optical path length changing unit arranged to change at least one of an optical path length of the reference light and an optical path length of the measurement light, the control method comprising
using a plurality of components relating to a plurality of signals obtained through the conversion of a plurality of interference signals that are obtained without changing the optical path length by the optical path length changing unit and of which intensities of folding components differ to generate a tomographic image of the object to be inspected.

15. A non-transitory computer-readable medium having stored thereon a program for causing a computer to perform the control method for an ophthalmic imaging apparatus according to claim 12.

16. A non-transit computer-readable medium having stored thereon a program for causing a computer to perform the control method for an ophthalmic imaging apparatus according to claim 13.

17. A non-transit computer-readable medium having stored thereon a program for causing a computer to perform the control method for an ophthalmic imaging apparatus according to claim 14.

18. The ophthalmic imaging apparatus according to claim 1, wherein:
the converter is a converter common to obtainment of the plurality of signals; and
the Nyquist frequency is a Nyquist frequency common the obtainment of the plurality of signals.

19. The ophthalmic imaging apparatus according to claim 4, wherein:
the converter including
a first converter arranged to convert the detected interference signal and
a second converter arranged to convert the detected interference signal, the second converter being different from the first converter; and
the arithmetic processing unit obtains any of the plurality of components by subtraction of the plurality of signals including a first signal obtained through the conversion by the first converter and a second signal obtained through the conversion by the second converter.

20. The ophthalmic imaging apparatus according to claim 10, wherein
the arithmetic processing unit uses the plurality of signals to obtain the plurality of components of which folding components differ, and uses the plurality of obtained components to generate the tomographic image.
